(19) United States
(12) Patent Application Publication
(10) Pub. No.: US 2008/0242966 A1
(43) Pub. Date: Oct. 2, 2008

(54) AIR PULSE DISCHARGING DEVICE FOR NONCONTACT TYPE TONOMETER AND NONCONTACT TYPE TONOMETER HAVING THE SAME

(76) Inventor: Hideo Sagehashi, Tokyo (JP)

Correspondence Address: DICKSTEIN SHAPIRO LLP, 1825 EYE STREET NW, Washington, DC, 20006-5403, US

(21) Appl. No.: 12/037,666

(22) Filed: Feb. 26, 2008

(30) Foreign Application Priority Data
Mar. 27, 2007 (JP) 2007-081758

(51) Int. Cl. A61B 3/16; F04B 53/12

(52) U.S. Cl. 600/401; 417/551

(57) ABSTRACT

An air pulse discharging device for a noncontact type tonometer, which includes: a cylinder; a piston arranged reciprocatably in the cylinder and which compresses air in a compression space; a nozzle which discharges the compressed air to an examined eye; at least one opening provided on the piston and which communicates the compression space with a cylinder atmospheric-air releasing chamber; and a valve disk provided to the piston and which opens and closes the at least one opening, the valve disk closes the at least one opening based on a differential pressure between the compression space and the cylinder atmospheric-air releasing chamber when the piston is driven to a direction of compressing the air in the compression space, and opens the at least one opening based on the differential pressure when the piston is driven to a direction opposite to the direction of compressing the air in the compression space.

### PRIORITY CLAIM

The present application is based on and claims priority from Japanese Application Number 2007-081758, filed Mar. 27, 2007, the disclosure of which is hereby incorporated by reference herein in its entirety.

### BACKGROUND

1. Field of the Invention

This invention relates to an air pulse discharging device used for a noncontact type tonometer, in which air in a compression space inside a cylinder is compressed and is discharged to a cornea of an examined eye, and to a noncontact type tonometer having the air pulse discharging device.

2. Description of the Related Art

For example, Japanese Patent publication number H07-16212, discloses an air pulse discharging device for a noncontact type tonometer. The disclosed device has a check valve which allows air to pass to the direction of an air discharge and prohibits air to pass to the opposite direction between a cylinder and a chamber, where a nozzle is provided.

In this air pulse discharging device, a solenoid valve is provided for a cylinder. The solenoid valve is used for a preparation for a next intraocular pressure measurement after the measurement in order to introduce outer air into a compression space of the cylinder. The solenoid valve is open when a piston returns to a default position inside the cylinder, and outer air is introduced into the compression space as the volume of the compression space is increased. In this case, as described above, air passes to the air discharge direction and to the opposite direction are prohibited by the check valve, so that it is possible to prevent outer air through the nozzle from being taken in the chamber, because a negative pressure occurs by increase of the volume of the compression space by move of a piston to the default position when the piston returns to the default position inside the cylinder. This makes it possible for the mist, formed by dispersed tears from an examined eye by discharged air to the examined eye, to prevent from being taken in the chamber through the nozzle with outer air when the piston moves to the default position. Therefore, it is possible to prevent the mist of the tears from sticking to a part of optical components structuring the chamber such as a chamber window and so on.

However, a structure, such as introducing outer air into the compression space, of a conventional air pulse discharging device including the device disclosed in JP-H07-16212 is generally complicated, because it is necessary to open the solenoid valve in order to introduce outer air into the compression space. In addition, it takes a preparation time for a next measurement because it is also necessary to have time to start operating the solenoid valve.

### SUMMARY

An object of the invention is to provide an air pulse discharging device for a noncontact type tonometer which has a simple structure, which is possible to shorten a preparation time for a next measurement and which is also possible to prevent mists such as atomized tears and so on from being taken in a compression space through a nozzle with air, and a noncontact type tonometer having the air pulse discharging device.

An air pulse discharging device for a noncontact type tonometer according to the present invention, includes a cylinder having a compression space and a cylinder atmospheric-air releasing chamber which is open to air, a piston arranged reciprocatably in the cylinder and which compresses air in the compression space, a nozzle which discharges the air compressed by the piston to an examined eye, at least one opening provided on the piston and which communicates the compression space with the cylinder atmospheric-air releasing chamber, and a valve disk provided to the piston and which opens and closes the at least one opening, the valve disk closes the at least one opening based on a differential pressure between the compression space and the cylinder atmospheric-air releasing chamber when the piston is driven to a direction of compressing the air in the compression space, and opens the at least one opening based on the differential pressure when the piston is driven to a direction opposite to the direction of compressing the air in the compression space.

Preferably, the piston has a bottomed cylindrical shape having a disk-shaped bottom, the valve disk has a disk shape, and at least one opening includes plural openings which are provided at even intervals on the bottom of the piston in a circumferential direction of the piston.

Preferably, opening areas of the openings are so set that sum of the opening areas of the openings becomes larger than an opening area of the nozzle.

Preferably, the valve disk is fixed to the piston in a peripheral part of the valve disk in such a manner that the at least one opening is openable and closeable.

Preferably, the valve disk is fixed to the piston in a central part of the valve disk in such a manner that the at least one opening is openable and closeable and a pressing member extending in a radial direction of the valve disk is provided between the valve disk and the piston.

Preferably, the valve disk includes a synthetic resin material.

Preferably, the piston includes a synthetic resin material.

An air pulse discharging device for a noncontact type tonometer according to the present invention, includes a cylinder having a compression space and a cylinder atmospheric-air releasing chamber which is open to air, a piston arranged reciprocatably in the cylinder and which compresses air in the compression, a chamber communicated through a communicating tube with the compression space, a chamber window structuring the chamber, a nozzle provided in the chamber in such a manner as to face a cornea of an examined eye and which discharges the air compressed by the piston to the examined eye, an optical system provided on a back side in an axial direction of the nozzle for observing an anterior eye part image of the examined eye through the chamber window, at least one opening provided on the piston and which communicates the compression space with the cylinder atmospheric-air releasing chamber, and a valve disk provided on the piston and which opens and closes the at least one opening, the valve disk closes the at least one opening based on a differential pressure between the compression space and the cylinder atmospheric-air releasing chamber when the piston is driven to a direction of compressing the air in the compression space, and the piston is driven to a direction of compressing the air in the compressing space, and opens the at least one opening based on the differential pressure when the piston is driven to a direction opposite to the direction of compressing the air in the compression space.

Preferably, the piston has a bottomed cylindrical shape having a disk-shaped bottom, the valve disk has a disk shape, and at least one opening includes plural openings which are provided at even intervals on the bottom of the piston in a circumferential direction of the piston.

Preferably, opening areas of the openings are so set that sum of the opening areas of the openings becomes larger than an opening area of the nozzle.

Preferably, the valve disk is fixed to the piston in a peripheral part of the valve disk in such a manner that the at least one opening is openable and closeable.

Preferably, the valve disk is fixed to the piston in a central part of the valve disk in such a manner that the at least one opening is openable and closeable and a pressing member extending in a radial direction of the valve disk is provided between the valve disk and the piston.

Preferably, the valve disk includes the synthetic resin material.

Preferably, the piston includes the synthetic resin material.

The present invention provides a noncontact type tonometer including any one of the air pulse discharging devices described above.

### BRIEF DESCRIPTION OF THE DRAWINGS

FIG. 1 is a schematic cross-sectional view of an air pulse discharging device for a noncontact type tonometer according to a first embodiment of the invention, in which a piston is at a bottom dead center position.

FIG. 2 is a schematic cross-sectional view of the air pulse discharging device for the noncontact type tonometer according to the first embodiment of this invention, in which the piston is at a top dead center position.

FIG. 3 is a cross-sectional view of FIG. 2 taken along the line A-A.

FIG. 4 is a schematic cross-sectional view of the air pulse discharging device for the noncontact type tonometer according to the first embodiment of the invention, in which the piston moves from the top dead center position on the way to the bottom dead center position.

FIG. 5 is a schematic cross-sectional view of an air pulse discharging device for a noncontact type tonometer according to a second embodiment of the invention, in which the piston is at the bottom dead center position.

FIG. 6 is a schematic of cross-sectional view of the air pulse discharging device for the noncontact type tonometer according to the second embodiment of the invention, in which the piston is at the top dead center position.

FIG. 7 is a cross-sectional view of FIG. 6 taken along the line B-B.

### DETAILED DESCRIPTION OF THE PREFERRED EMBODIMENTS

Hereinafter, referring to the drawings, an air pulse discharging device for a noncontact type tonometer according to some embodiments of the invention will be explained.

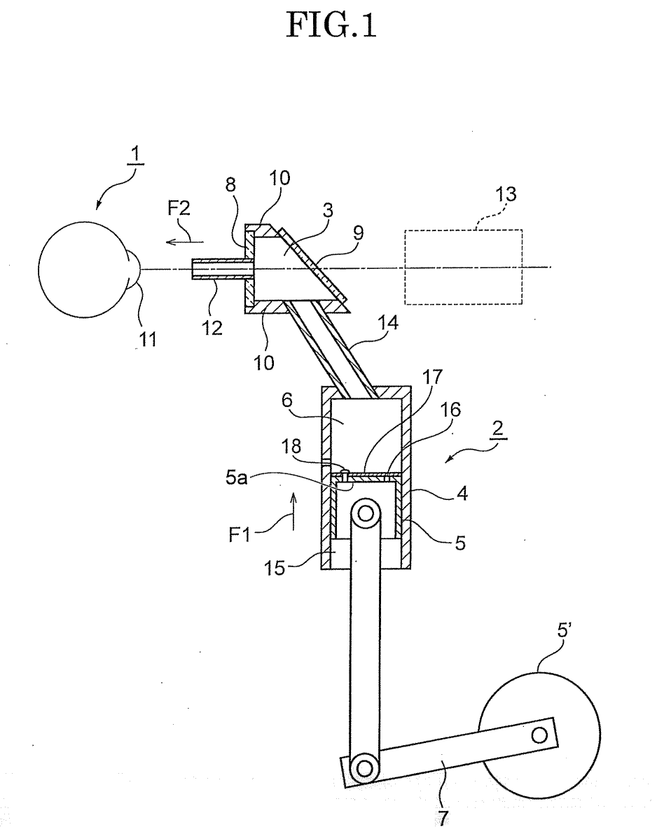

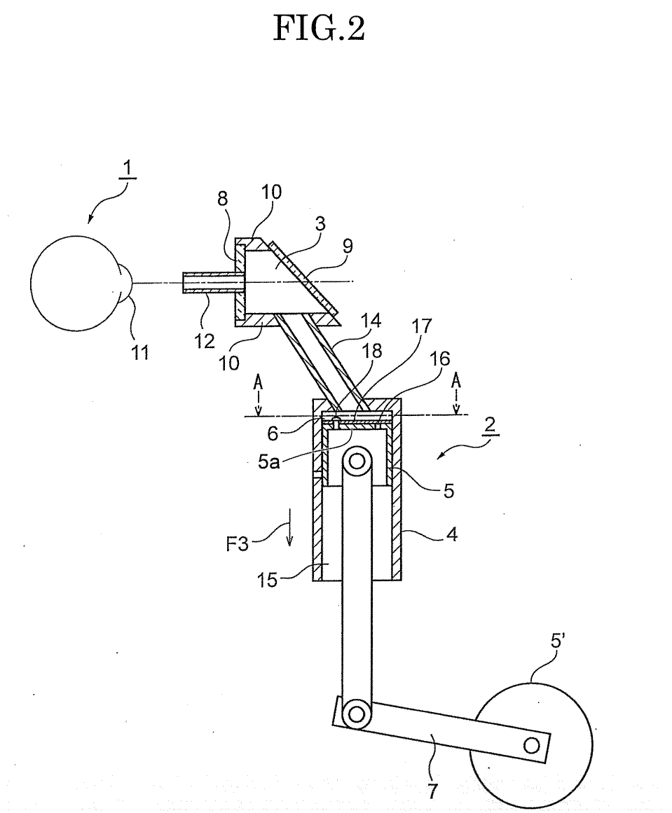

From FIGS. 1 to 4, they are schematic diagrams of an air pulse discharging device for a noncontact type tonometer according to a first embodiment of the invention. In FIGS. 1 and 2, a numeral 1 denotes an examined eye and a numeral 2 denotes an air discharging device.

The air discharging device 2 has a chamber 3, a cylinder 4 and a rotary solenoid 5′. A piston 5 is provided reciprocatably in the cylinder 4. The piston 5 has a cylindrical shape, and has a disk-shaped bottom, i.e. a top part 5a, which structures the bottom of the bottomed cylinder. The piston 5 and the cylinder 4 structure a compression space 6.

The piston 5 is connected by a rod 7 with the rotary solenoid 5′. The piston 5 reciprocates inside the cylinder 4, along an axial direction of the cylinder 4, that is, along an up-and-down direction, by a rotary reciprocating motion of the rotary solenoid 5′.

The chamber 3 generally includes a cover glass 8, a chamber window 9 and a partition wall 10. The cover glass 8 has a nozzle 12 extending from the cover glass 8 to the outside of the chamber 3. A tip of the nozzle 12 faces a cornea 11 of the examined eye 1, when measuring an intraocular pressure of the examined eye 1.

Regarding the chamber 3, at the opposite side of the examined eye 1, that is, at the back side of an axial direction of the nozzle 12, there is an optical system 13 which observes an anterior eye part image of the examined eye 1 through the chamber window 9.

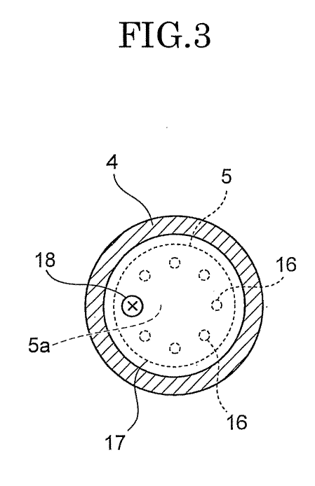

The chamber 3 and the compression space 6 are mutually communicated through a communicating tube 14. The top part 5a of the piston 5 is formed with plural openings 16. The openings 16 are communicated the compression space 6 with a cylinder atmospheric-air releasing chamber 15, which is communicated with air. Each opening, as illustrated in FIG. 3, is formed at even intervals on the bottom of the piston in circumferential directions of the piston 5. It is preferable to set number of the openings 16 and those opening areas respectively, so that sum of opening areas of the openings 16 becomes larger than an opening area of the nozzle 12.

The piston 5 is provided with a valve disk 17, which opens and closes each opening 16.

The valve disk 17, as illustrated in FIG. 3, for example, is a disk-shaped synthetic resin plate and is arranged to cover the top part 5a at the top of the piston 5. Here, the valve disk 17 is fixed at the top part 5a of the piston 5 around a peripheral part of the valve disk 17 by a screw 18. The piston 5 also can be formed by a synthetic resin. In that case, each of resin materials for the valve disk 17 and for the piston 5 may set to be different.

The valve disk 17 closes the openings 16 based on a differential pressure between the compression space 6 and the cylinder atmospheric-air releasing chamber 15 when the piston 5 is driven to the direction of compressing air in the compression space 6, and opens the openings 16 based on a differential pressure between the compression space 6 and the cylinder atmospheric-air releasing chamber 15 when the piston 5 is driven to the opposite direction of compressing air in the compression space 6.

Next, the functions of the air pulse discharging device according to the first embodiment of the invention will be explained.

In FIG. 1, the piston 5 is at the bottom dead center position and it is just before when the piston 5 is driven by the rotary solenoid 5′. In this state, the valve disk 17 closes openings 16. When the piston 5 is driven to the direction of an arrow F1, the volume of the compression space 6 decreases. This makes a pressure of the compression space 6 rise. Along with a rise of the pressure inside this compression space 6, air inside the chamber 3 is discharged, as an air pulse, from the nozzle 12 to the cornea 11 of the examined eye 1 as illustrated as an arrow F2. An intraocular pressure measurement is accomplished by an air discharge from this nozzle 12. As principles of intraocular pressure measurement are well-known, explanations will not be described in detail.

In FIG. 2, it illustrates that the piston 5 is at the top dead center position. In this state, the driving of the rotary solenoid 5′ is stopped. This makes the piston 5 move, for example, to the direction of falling to the bottom dead center position because of its own weight, that is, to the direction of an arrow F3.

When the piston 5 moves to the bottom dead center position, an air pressure at the front side of moving direction increases along with the move of the piston 5. In other words, an air pressure of the cylinder atmospheric-air releasing chamber 15 increases. On the other hand, an air pressure inside the compression space 6 attempts to be a negative pressure along with increase of the volume of the compression space 6 at the back side of moving direction of the piston 5.

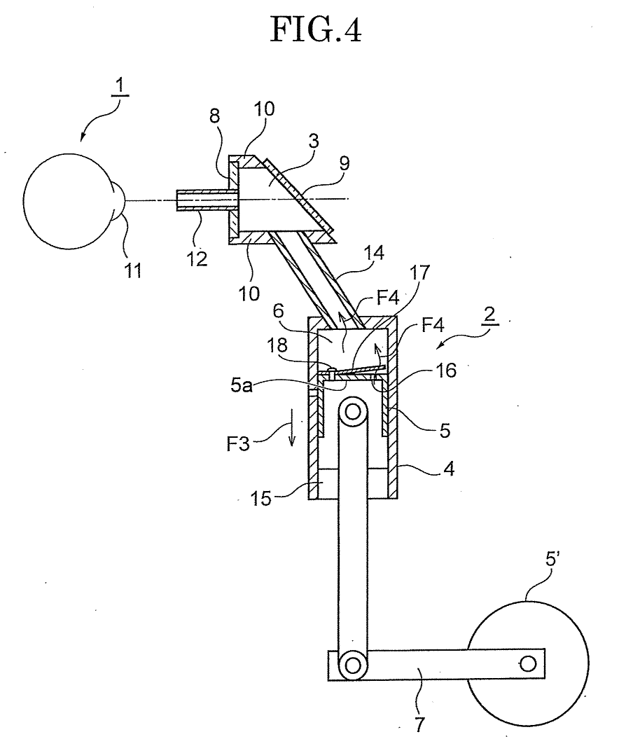

The valve disk 17 deforms based on the differential pressure between a pressure increase of this cylinder atmospheric-air releasing chamber 15 and a pressure attenuation of the compression space 6, that makes each opening 16 open respectively. When each opening 16 opens respectively, an airflow, from the cylinder atmospheric-air releasing chamber 15 to the compression space 6, which is in the direction of an arrow F4, occurs as illustrated in FIG. 4. This makes an introduction of air from the nozzle 12 into the compression space 6 by the negative pressure, which occurs in accordance with the volume increase of the compression space, to be decreased significantly. Therefore, it prevents the mist such as atomized tears and so on from being taken in the compression space 6 through the nozzle 12.

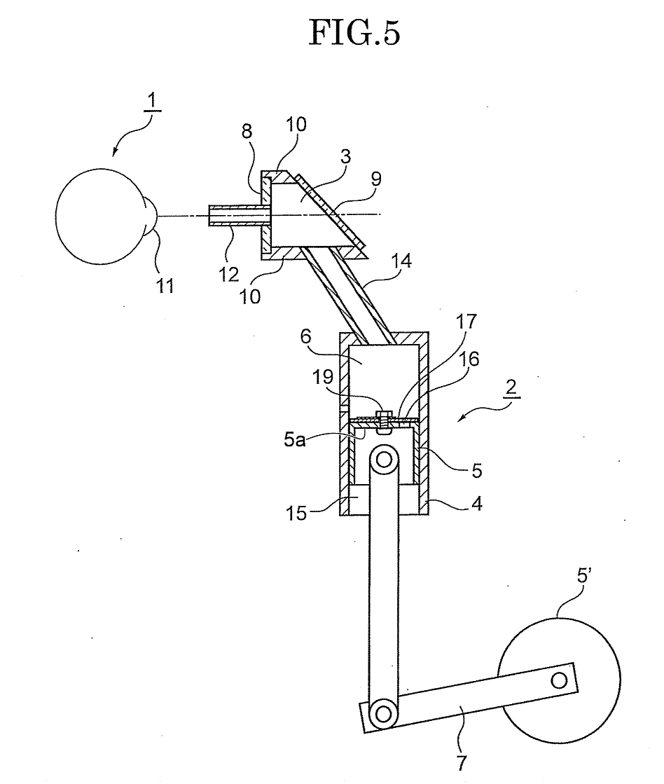

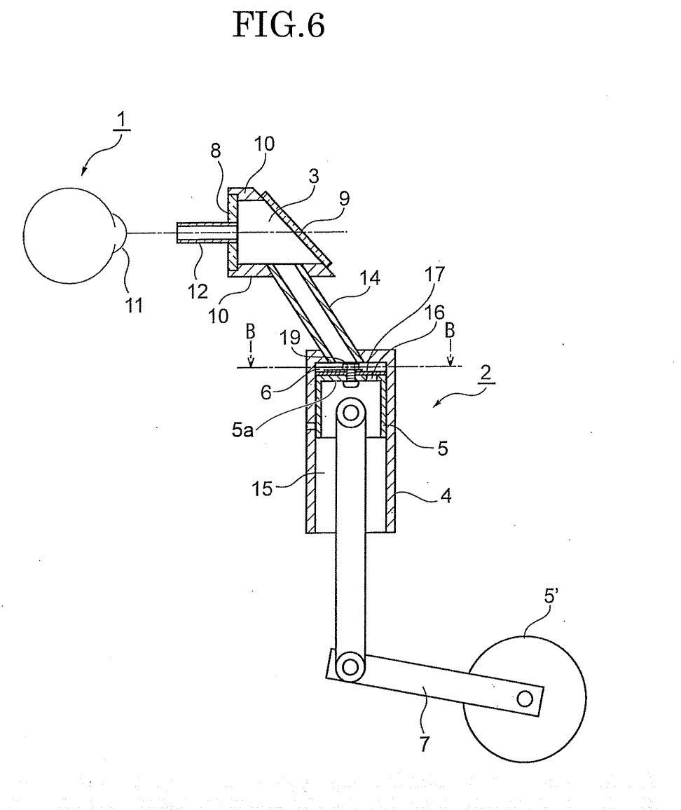

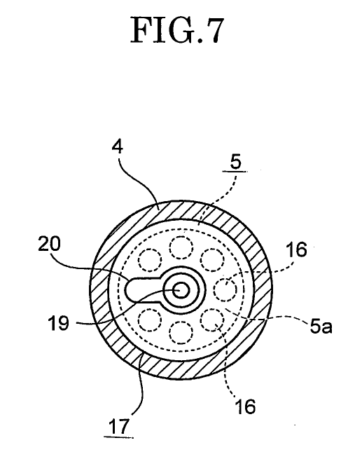

From FIGS. 5 to 7, they are schematic diagrams of an air pulse discharging device for a noncontact type tonometer according to a second embodiment of the invention.

Here, the valve disk 17 is fixed on the top part 5a of the piston 5 in its center by a screw 19, so that each opening is openable and closeable. A pressing member 20 extending to a radial direction, is interposed between the valve disk 17 and the piston 5. The radial direction part of the valve disk 17 is fixed partially by this pressing member 20.

The functions of this air pulse discharging device for the noncontact type tonometer according to the second embodiment are almost same as the functions of the first embodiment. Therefore, explanations of the functions of the second embodiment will not be described in detail.

According to the air pulse discharging device for the noncontact type tonometer according to the embodiments of the invention described above, the openings which connect through the compression space inside the cylinder and the cylinder atmospheric-air releasing chamber, are formed on the piston. The piston is provided with a valve disk. The valve disk closes the openings based on a differential pressure between the compression space and the cylinder atmospheric-air releasing chamber when the piston is driven to the direction of compressing air in the compression space, and opens the openings based on a differential pressure between the compression space and the cylinder atmospheric-air releasing chamber when the piston is driven to the opposite direction of compressing air in the compression space. This allows the openings to be closed and air to be discharged efficiently to an examined eye when air is discharged to the examined eye.

In addition, when air is introduced to the compression space, the openings are open based on a differential pressure between the pressure increase at the front side of moving direction and the pressure attenuation of the compression space at the back side of moving direction along with a move of the piston, which makes an airflow from the cylinder atmospheric-air releasing chamber to the compression space occur. This allows air to be introduced to the compression space. This makes an introduction of air from the nozzle into the compression space by the negative pressure, which occurs in accordance with the volume increase of the compression space, to be decreased significantly.

Therefore, it prevents the mists such as atomized tears and so on from being taken in the compression space through the nozzle. Additionally, it is possible to simplify a structure and to shorten a preparation time for a next measurement, comparing to an operation of conventional solenoid valves to drive to introduce outer air to the compression space.

Although the present invention has been described in terms of exemplary embodiments, it is not limited thereto. It should be appreciated that variations may be made in the embodiments described by persons skilled in the art without departing from the scope of the present invention as defined by the following claims.

1. An air pulse discharging device for a noncontact type tonometer, comprising:
a cylinder having a compression space and a cylinder atmospheric-air releasing chamber which is open to air;
a piston arranged reciprocatably in the cylinder and which compresses air in the compression space;
a nozzle which discharges the air compressed by the piston to an examined eye;
at least one opening provided on the piston and which communicates the compression space with the cylinder atmospheric-air releasing chamber; and
a valve disk provided to the piston and which opens and closes the at least one opening, the valve disk closes the at least one opening based on a differential pressure between the compression space and the cylinder atmospheric-air releasing chamber when the piston is driven to a direction of compressing the air in the compression space, and opens the at least one opening based on the differential pressure when the piston is driven to a direction opposite to the direction of compressing the air in the compression space.

2. An air pulse discharging device according to claim 1, wherein the piston has a bottomed cylindrical shape having a disk-shaped bottom, the valve disk has a disk shape, and at least one opening includes plural openings which are provided at even intervals on the bottom of the piston in a circumferential direction of the piston.

3. An air pulse discharging device according to claim 2, wherein opening areas of the openings are so set that sum of the opening areas of the openings becomes larger than an opening area of the nozzle.

4. An air pulse discharging device according to claim 1, wherein the valve disk is fixed to the piston in a peripheral part of the valve disk in such a manner that the at least one opening is openable and closeable.

5. An air pulse discharging device according to claim 1, wherein the valve disk is fixed to the piston in a central part of the valve disk in such a manner that the at least one opening is openable and closeable and a pressing member extending in a radial direction of the valve disk is provided on the valve disk.

6. An air pulse discharging device according to claim 1, wherein the valve disk includes a synthetic resin material.

7. An air pulse discharging device according to claim 1, wherein the piston includes a synthetic resin material.

8. An air pulse discharging device for a noncontact type tonometer, comprising:
a cylinder having a compression space and a cylinder atmospheric-air releasing chamber which is open to air;
a piston arranged reciprocatably in the cylinder and which compresses air in the compression;
a chamber communicated through a communicating tube with the compression space;
a chamber window structuring the chamber;
a nozzle provided in the chamber in such a manner as to face a cornea of an examined eye and which discharges the air compressed by the piston to the examined eye; an optical system provided on a back side in an axial direction of the nozzle for observing an anterior eye part image of the examined eye through the chamber window;
at least one opening provided on the piston and which communicates he compression space with the cylinder atmospheric-air releasing chamber; and
a valve disk provided on the piston and which opens and closes the at least one opening, the valve disk closes the at least one opening based on a differential pressure between the compression space and the cylinder atmospheric-air releasing chamber when the piston is driven to a direction of compressing the air in the compression space, and the piston is driven to a direction of compressing the air in the compressing space, and opens the at least one opening based on the differential pressure when the piston is driven to a direction opposite to the direction of compressing the air in the compression space.

9. An air pulse discharging device according to claim 8, wherein the piston has a bottomed cylindrical shape having a disk-shaped bottom, the valve disk has a disk shape, and at least one opening includes plural openings which are provided at even intervals on the bottom of the piston in a circumferential direction of the piston.

10. An air pulse discharging device according to claim 9, wherein opening areas of the openings are so set that sum of the opening areas of the openings becomes larger than an opening area of the nozzle.

11. An air pulse discharging device according to claim 8, wherein the valve disk is fixed to the piston in a peripheral part of the valve disk in such a manner that the at least one opening is openable and closeable.

12. An air pulse discharging device according to claim 8, wherein the valve disk is fixed to the piston in a central part of the valve disk in such a manner that the at least one opening is openable and closeable and a pressing member extending in a radial direction of the valve disk is provided on the valve disk

13. An air pulse discharging device according to claim 8, wherein the valve disk includes the synthetic resin material.

14. An air pulse discharging device according to claim 8, wherein the piston includes the synthetic resin material.

15. A noncontact type tonometer, comprising the air pulse discharging device according to claim 1.

16. A noncontact type tonometer, comprising the air pulse discharging device according to claim 8.
